(19) United States
(12) Patent Application Publication
(10) Pub. No.: US 2006/0009506 A1
(43) Pub. Date: Jan. 12, 2006

(54) Drugs for the treatment of neoplastic disorders

(75) Inventors: John K. Westwick, San Ramon, CA (US); Helen Yu, Mountain View, CA (US); Stephen Owens, Martinez, CA (US); Marnie L. MacDonald, Pleasnaton, CA (US)

Correspondence Address: Isaac A. Angres;Suite 301, 2001 Jefferson Davis Highway, Arlington, VA, 22202, US

(73) Assignee: Odyssey Thera, Inc., San Ramon, CA (US)

(21) Appl. No.: 11/174,630

(22) Filed: Jul. 6, 2005

Related U.S. Application Data
(60) Provisional application No. 60/586,235, filed on Jul. 9, 2004.

(51) Int. Cl. A61K 31/4172

(52) U.S. Cl. 514/395

(57) ABSTRACT

The invention features a method for treating a patient having a cancer or other neoplasm, by administering to the patient one of the following drugs or a metabolite or analog thereof: cinnarizine; desipramine; fenofibrate; flunarizine; isoreserpine; nicardipine; promazine; promethazine; suloctidil; terfenadine; atorvastatin; mebeverine; sertraline; albendazole; bepridil; bergaptene; clomiphene; dichlorophene; droperidol; mebendazole; meclocycline; metergoline; ramiphenazone; sanguinarine; dipyrone; nicardipine; or 4-dimethylaminoantipyrine.

This application claims the priority benefit under 35 U.S.C. section 119 of U.S. Provisional Patent Application No. 60/586,235 entitled “Drugs for the treatment of neoplastic disorders”, filed Jul. 9, 2004, which is in its entirety herein incorporated by reference.

### BACKGROUND OF THE INVENTION

The invention relates to the treatment of neoplastic disorders such as cancer.

Cancer is a disease marked by the uncontrolled growth of abnormal cells. The abnormal cells undergo hyperproliferation and may invade and metastasize to other organs. There are 100 different types of cancers in man.

Lung cancer is the most prevalent cancer-related cause of death. It is the second most commonly occurring cancer among men and women. Cancers that begin in the lungs are divided into two major types, non-small cell lung cancer and small cell lung cancer, depending on their cell of origin. Non-small cell lung cancere (squamous cell carcinoma, adenocarcinoma, and large cell carcinoma) generally spreads to other organs more slowly than does small cell lung cancer. Small cell lung cancer is the less common type, accounting for about 20% of all lung cancer.

Other cancers include brain cancer, breast cancer, cervical cancer, colon cancer, gastric cancer, kidney cancer, leukemia, liver cancer, lymphoma, ovarian cancer, pancreatic cancer, prostate cancer, rectal cancer, sarcoma, skin cancer, testicular cancer, and uterine cancer. These cancers, like lung cancer, are sometimes treated with chemotherapy. Certain cancers, including as pancreatic cancer, ovarian cancer, and skin cancer (melanoma) are characterized by rapid spread of the disease and a relative paucity of targeted therapies.

### SUMMARY OF THE INVENTION

We have discovered that cinnarizine; desipramine; fenofibrate; flunarizine; reserpine; isoreserpine; nicardipine; promazine; promethazine; suloctidil; terfenadine; atorvastatin; mebeverine; sertraline; albendazole; bepridil; bergaptene; clomiphene; dichlorophene; droperidol; mebendazole; meclocycline; metergoline; ramiphenazone; sanguinarine; dipyrone; nicardipine; 4-dimethylaminoantipyrine; exhibit substantial antiproliferative activity against cancer cells.

Structural and functional analogs of each of these compounds are known, and any of these analogs can be used in the antiproliferative compositions and methods of the invention. Metabolites of the abovementioned drugs are also known. Many of these metabolites share one or more biological activities with the parent compound and, accordingly, can also be used in the antiproliferative compositions and methods of the invention. In addition we have discovered that the natural products neriifolin, peruvoside, tomatine, beta-lapachone and niclosamide have substantial antiproliferative activity against human tumor cells. Accordingly, the invention features a method for treating a mammal patient having a cancer or other neoplasm, by administering to the patient one of the above drugs or natural products in an amount sufficient to inhibit the growth of the neoplasm.

The cancer treated according to any of the methods of the invention, described below, can be lung cancer (squamous cell carcinoma, adenocarcinoma, or large cell carcinoma), brain cancer, breast cancer, cervical cancer, colon cancer, gastric cancer, kidney cancer, leukemia, liver cancer, lymphoma, ovarian cancer, pancreatic cancer, prostate cancer, rectal cancer, sarcoma, skin cancer, testicular cancer, or uterine cancer.

The invention also features methods for identifying compounds useful for treating a mammal patient having a neoplasm. The method includes the steps of contacting cancer cells in vitro with (i) cinnarizine; desipramine; fenofibrate; flunarizine; reserpine; isoreserpine; nicardipine; promazine; promethazine; suloctidil; terfenadine; atorvastatin; mebeverine; sertraline; albendazole; bepridil; bergaptene; clomiphene; dichlorophene; droperidol; mebendazole; meclocycline; metergoline; ramiphenazone; sanguinarine; dipyrone; nicardipine; or 4-dimethylaminoantipyrine; and determining whether the cancer cells grow more slowly than cancer cells that are untreated or are treated with a vehicle or an inactive compound.

### OBJECTS AND ADVANTAGES OF THE INVENTION

It is an object of the present invention to provide drugs useful for the treatment of cancer in mammals and more specifically man.

An additional object of the invention is to provide methods and compositions for the treatment of cancer and other neoplastic disorders.

An further object of the invention is to re-indicate the known pharmacopeia, that is, to provide new therapeutic uses for existing drugs.

An advantage of the invention is that many of the drugs discovered to have antiproliferative activity have already been shown to be safe and well-tolerated during chronic administration in man.

A further advantage of the invention is that one or more drugs, previously withdrawn from the market, may prove to be sufficiently safe and efficacious for use in the context of the oncology clinic.

### BRIEF DESCRIPTION OF THE DRAWINGS

FIG. 1 Antiproliferative activity of fenofibrate vs an inactive analog. The ability of fenofibrate to block proliferation of PC-3 cells is shown. An analog, WY-14643, is inactive in the PC-3 assay, demonstrating structure-activity relationships in these cellular assays. Dose dependence for fenofibrate (triplicate assays) is shown in the proliferation assay as compared to the DMSO control.

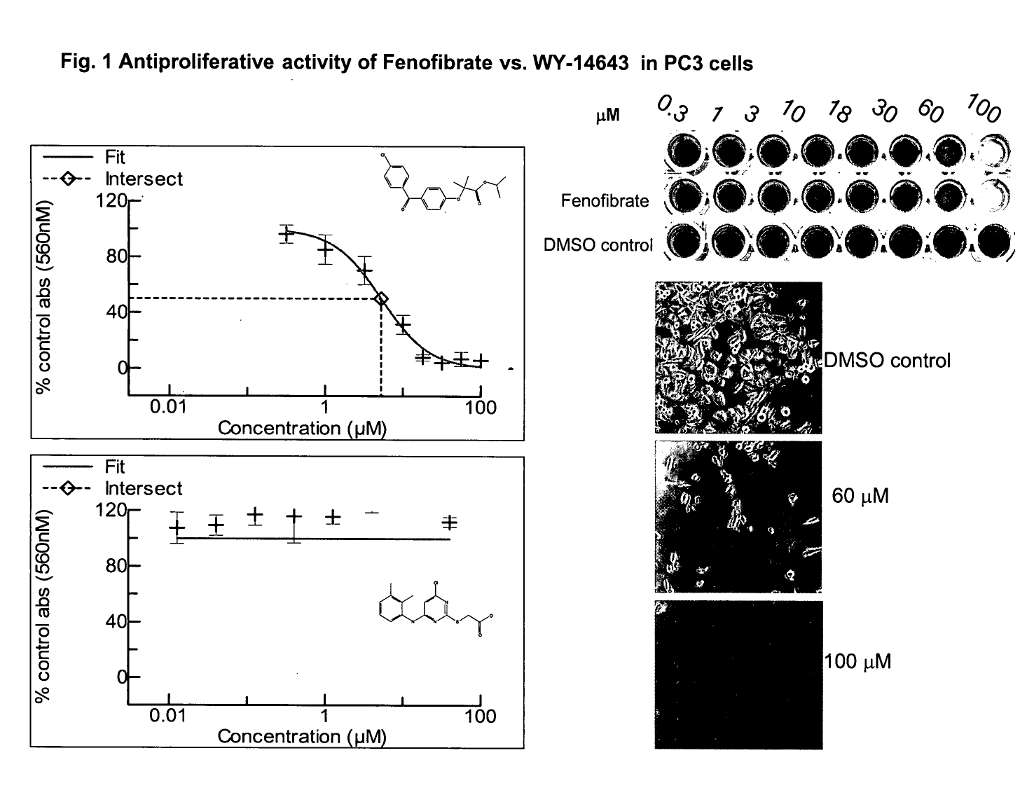

FIG. 2 Antiproliferative activity of sertraline (Zoloft)

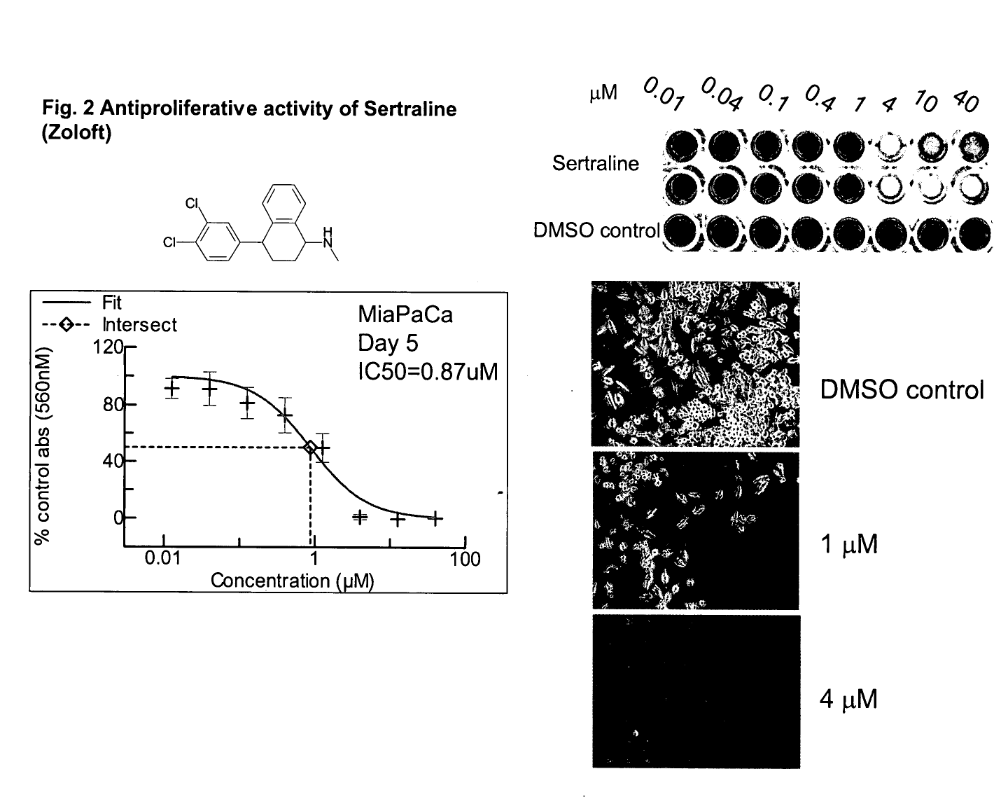

FIG. 3 Antiproliferative activity of cinnarizine

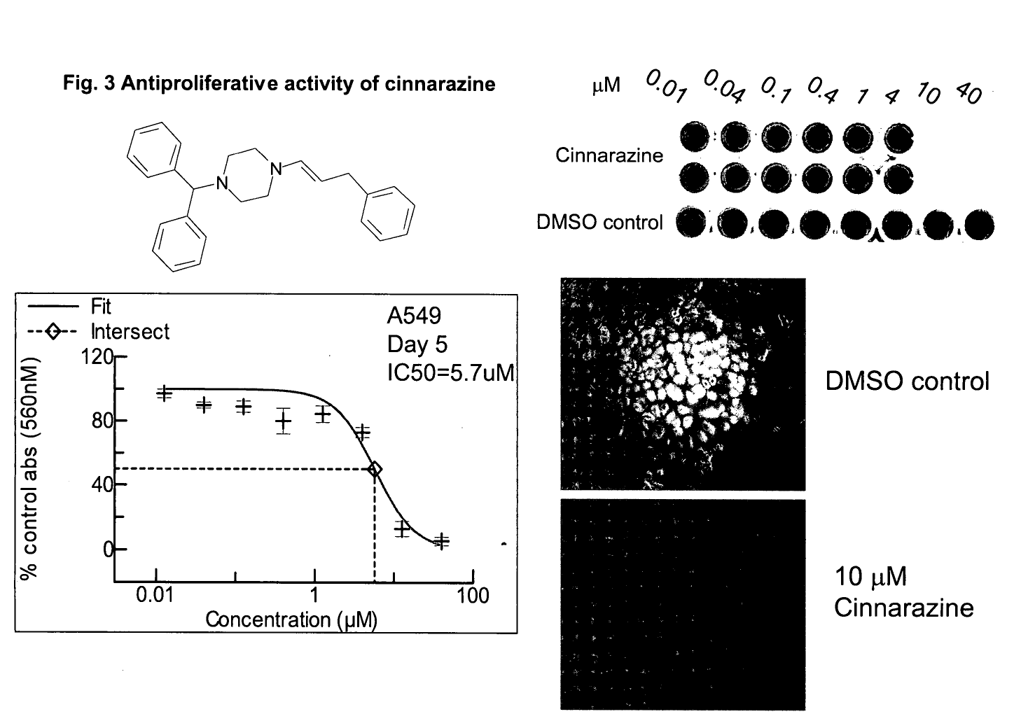

FIG. 4 Antiproliferative activity of isoreserpine

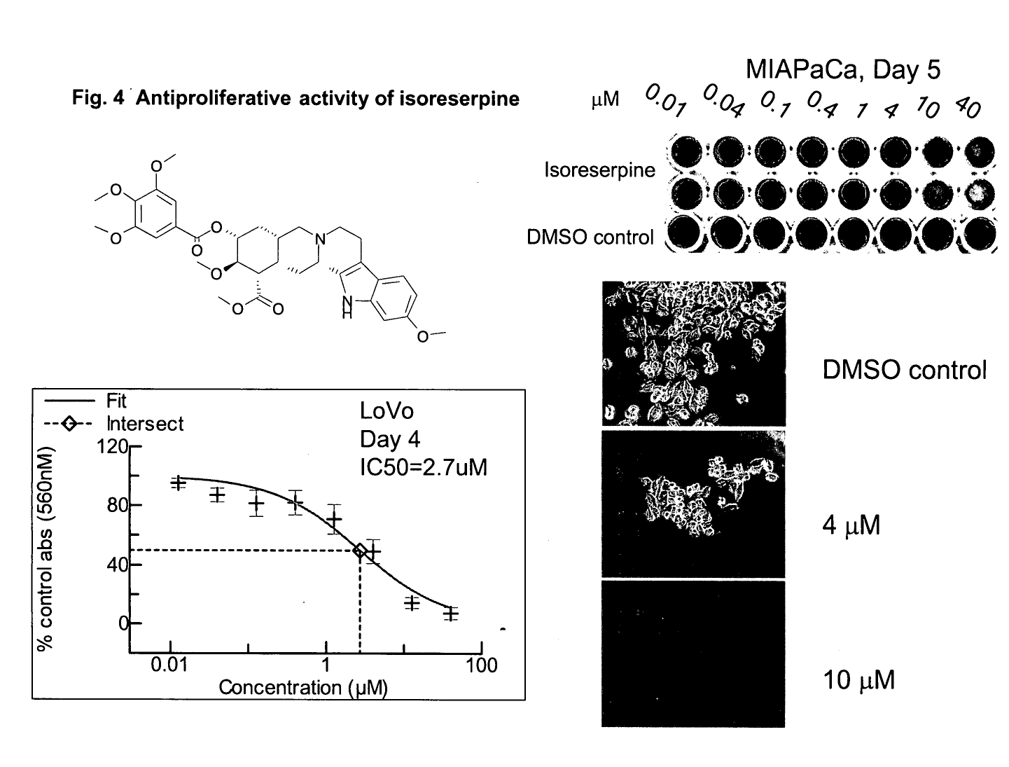

FIG. 5 Antiproliferative activity of clotrimazole

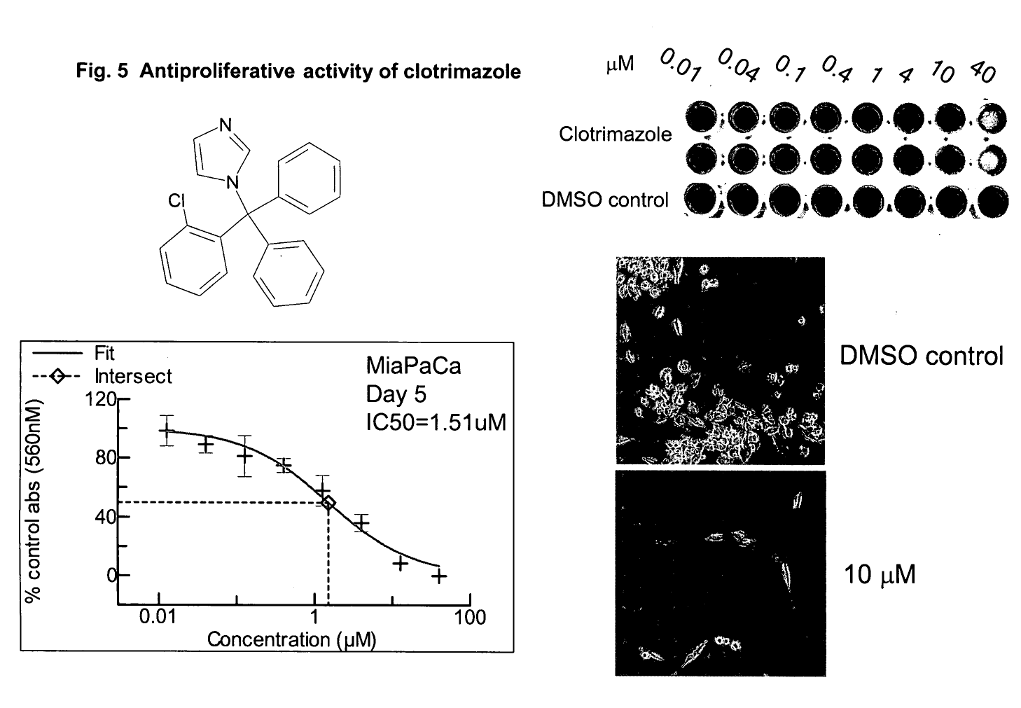

FIG. 6 Antiproliferative activity of terfenadine (Seldane)

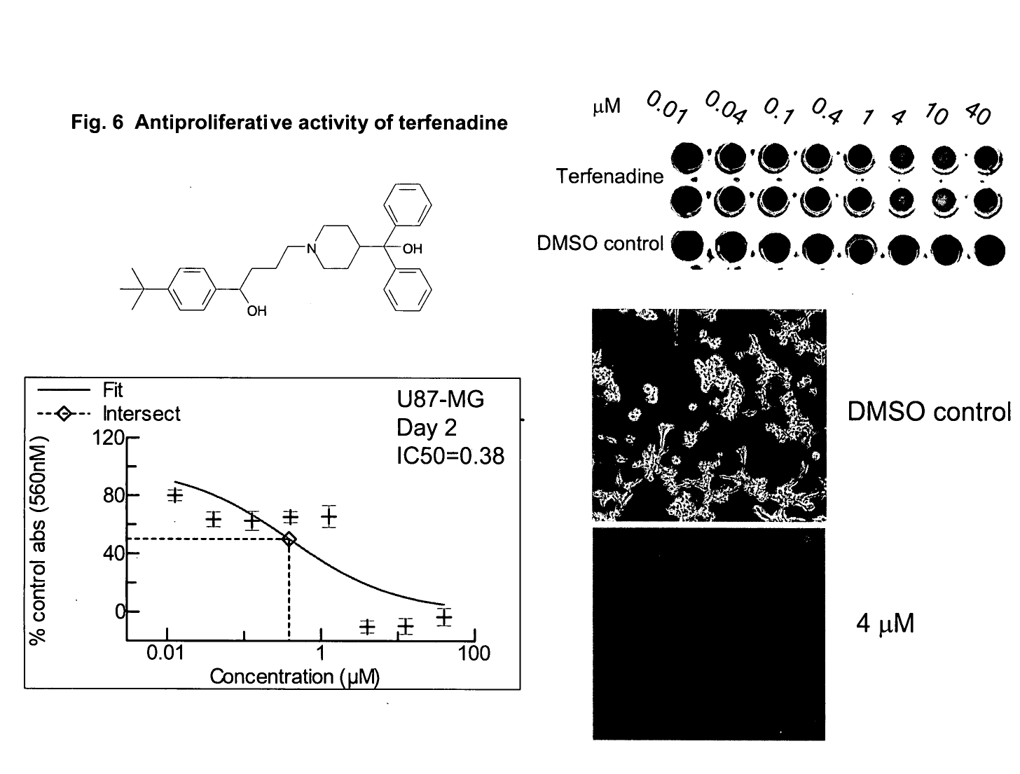

FIG. 7 Antiproliferative activity of atorvastatin (Lipitor)

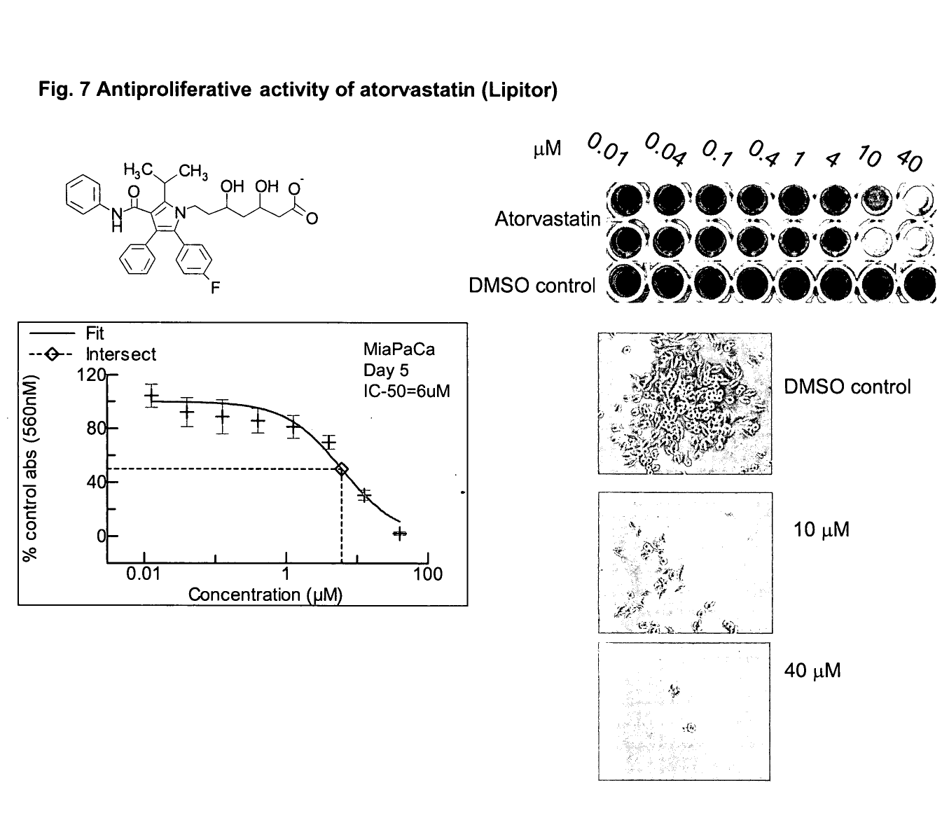

FIG. 8 Detailed comparison of the antiproliferative activities of nine different drugs in five different tumor cell lines. Drug concentrations giving half-maximal inhibition in proliferation assays, together with the time (days) at which the assays were performed following drug addition, are shown.

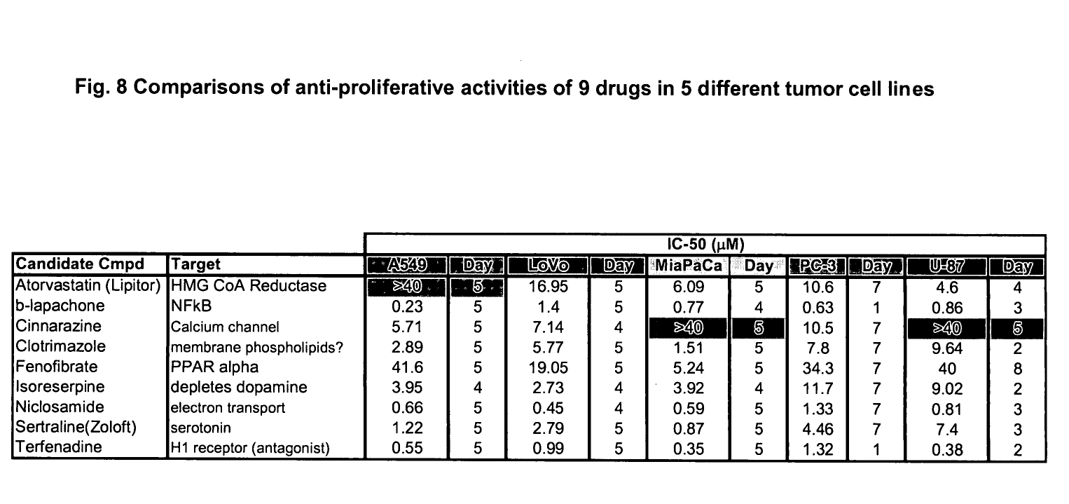

FIG. 9 Summary slide showing drugs discovered to have antiproliferative activity.

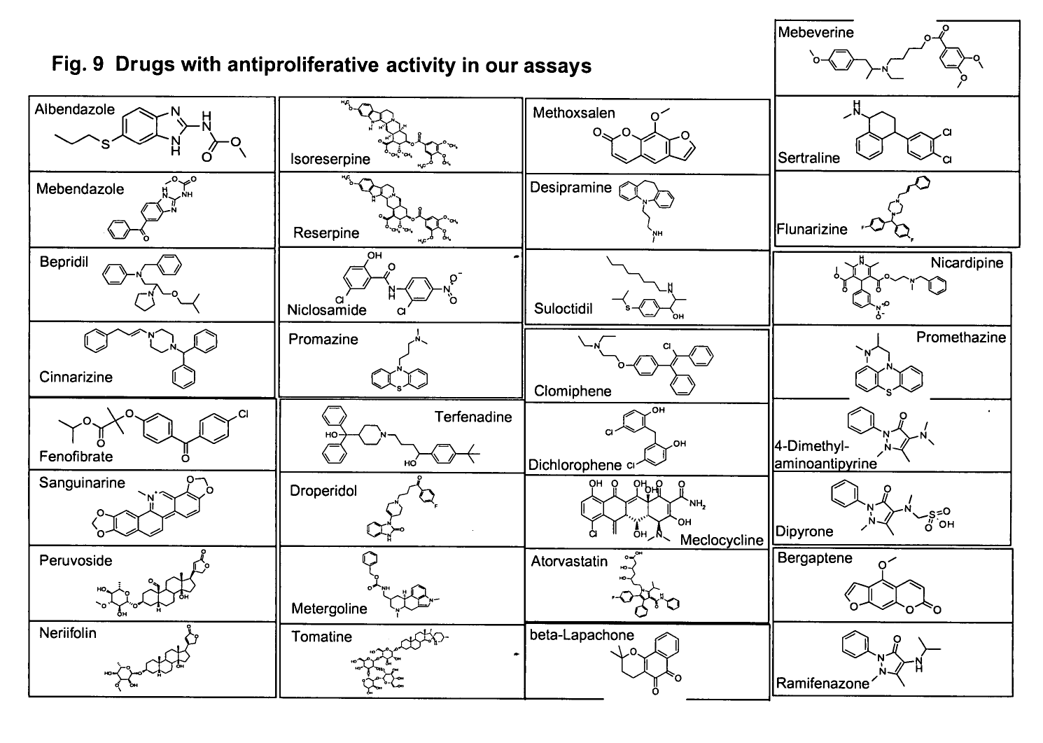

### DETAILED DESCRIPTION OF THE INVENTION

We have discovered that certain antihypertensive, antibacterial, antifungal, antipsychotic, antiemetic, cholesterol-lowering and lipid-lowering drugs have substantial antiproliferative activity against cancer cells. Most of the compounds are identified as having antiproliferative activity are known drugs that are either currently marketed for other indications (either as protected or generic drugs); were previously marketed and were either discontinued or withdrawn from the market. Concentrations that exhibit maximal antiproliferative activity against cancer cells are not toxic to normal cells. Moreover, in cases where plasma levels of the compounds have been documented in the literature, the concentrations demonstrating antiproliferative activity are consistent with achievable plasma levels in animals and/or in man.

The overall similarities in behavior of different cancer cells are a manifestation of common and essential alterations in cell physiology that collectively dictate malignant growth: self-sufficiency in growth signals, insensitivity to growth-inhibitory signals, evasion of programmed cell death (apoptosis), limitless replicative potential, sustained angiogenesis, and tissue invasion and metastasis. These acquired traits are shared by most—if not all—human cancer types. A successful therapeutic strategy would involve the discovery and development of drugs that are capable of modulating one or more of the intrinsic pathways that control the behavior of the cancer cell.

The strategy for discovering antiproliferative activities of drugs was as follows. We screened the known pharmacopeia and a select natural product library in order to identify drugs that are capable of modulating the activity of the oncogenic pathways underlying the cancer phenotype. By ‘known drug’ and ‘known pharmacopeia’ we mean drugs currently or previously used in man for indications other than oncology. We identified over 30 drugs with previously-unsuspected activity against ‘hallmark’ cancer pathways. We then showed that these drugs have anti-proliferative activity in tumor cell models, underscoring the utility and predictability of the approach. The strategy and methods presented herein represent an entirely novel strategy for therapeutic discovery. Importantly, the drugs we identified represent potential treatments for cancer in man.

In the full application we describe in more detail the current state of knowledge of each of the drugs, their analogs and metabolites.

Based on the known properties that are shared between these drugs and their analogs and metabolites, it is likely that structurally related compounds can be substituted for these compounds in the antiproliferative compositions and methods of the invention. Information regarding each of the drugs and its analogs and metabolites is provided herein. The formulas for each of the agents, its analogs and metabolites is also provided.

Assay for Activity of Compounds on Cancer Pathways in Human Cells

We first screened compounds derived from the the known pharmacopeia and from a selected natural product collection using two types of assays: (a) measurements of post-translational modifications of proteins; (b) measurements of protein-protein interactions. For the purpose of measuring post-translational modifications of proteins, we used immunofluorescence methods combined with phospho-specific antibodies. For the purpose of quantifying and localizing protein complexes we used fluorescence methods based on protein-fragment complementation assays (PCA). ‘Hits’ from the initial screens were then tested for antiproliferative activity in up to 5 different human tumor cell lines. First, drugs with significant activity in the screen(s) were tested for their ability to block proliferation of a human tumor cell line (PC-3) at an initial concentration of 10 micromolar. For drugs with antiproliferative activity in PC-3 cells, dose-response curves were generated to determine the EC50 for antiproliferative activity and additional tumor cell lines were tested to determine the breadth of activity. Methods for assay development, validation, screening, and testing of antiproliferative activity are described below.

Immunofluorescence Assays

Immunofluorescence was performed on drug-treated cells using antibodies that specifically recognize phosphorylated forms of key signaling proteins. HEK293T cells were seeded in black-walled, poly-lysine coated 96-well plates (Greiner) at a density of 30,000/well. After 24 hours, cells in duplicate wells were treated with drugs. 100 ng/ml hEGF added to the cells during the last 5 min of drug treatment. The cells were rinsed once with PBS and fixed with 4% formaldehyde for 10 min. The cells were subsequently permeabilized with 0.25% Triton X-100 in PBS and incubated with 3% BSA for 30 min to block non-specific antibody binding. Each of the 4 sets of identically treated cells were then incubated with rabbit antibodies against phosphorylated CREB (Ser133), hsp27 (Ser82), pERK (T202/Y204) (Cell Signaling), or BSA in PBS. The cells were rinsed with PBS and incubated with Alexa488 conjugated goat anti-rabbit secondary antibody (Molecular Probes). Cell nuclei were stained with Hoechst33342 (Molecular Probes). Images were acquired using Discovery-1 High Content Imaging System (Molecular Devices). Background fluorescence due to nonspecific binding by the secondary antibody was established with the use of cells that were incubated with BSA/PBS and without primary antibodies. Fluorescence intensities were quantitated using Image J-based image analysis algorithm as described below.

Protein-Fragment Complementation Assays

Reporter fragments for PCA were generated by oligonucleotide synthesis (Blue Heron Biotechnology, Bothell, Wash.). First, oligonucleotides coding for polypeptide fragments YFP[1]and YFP[2] (corresponding to amino acids 1-158 and 159-239 of YFP) were synthesized. Next, PCR mutagenesis was used to generate the mutant fragments IFP[1] and IFP[2]. The IFP[1] fragment corresponds to YFP[1]-(F46L, F64L, M153T) and the IFP[2] fragment corresponds to YFP[2]-(V163A, S175G). These mutations have been shown to increase the fluorescence intensity of the intact YFP protein (Nagai et al., Nature Biotechnology, 20, 87-90 2002). The YFP[1], YFP[2], IFP[1] and IFP[2] fragments were amplified by PCR to incorporate restriction sites and a linker sequence, described below, in configurations that would allow fusion of a gene of interest to either the 5′- or 3′-end of each reporter fragment. The reporter-linker fragment cassettes were subcloned into a mammalian expression vector (pcDNA3.1Z, Invitrogen) that had been modified to incorporate the replication origin (oriP) of the Epstein Barr virus (EBV). The oriP allows episomal replication of these modified vectors in cell lines expressing the EBNAI gene, such as HEK293E cells (293-EBNA, Invitrogen). Additionally, these vectors still retain the SV40 origin, allowing for episomal expression in cell lines expressing the SV40 large T antigen (e.g. HEK293T, Jurkat or COS). The integrity of the mutated reporter fragments and the new replication origin were confirmed by sequencing. PCA fusion constructs were prepared for a large number of proteins known to participate in cellular pathways (Table 1 and Table 2). The full coding sequence for each gene of interest was amplified by PCR from a sequence-verified full-length cDNA. Resulting PCR products were column purified (Centricon), digested with appropriate restriction enzymes to allow directional cloning, and fused in-frame to either the 5′ or 3′-end of YFP[1], YFP[2], IFP[1] or IFP[2] through a linker encoding a flexible 10 amino acid peptide (Gly.Gly.Gly.Gly.Ser)₂. The flexible linker ensures that the orientation/arrangement of the fusions is optimal to bring the reporter fragments into close proximity (Pelletier et al., Journal of Biomolecular Techniques, 10: 32-39 1998). Recombinants in the host strains DH5-alpha (Invitrogen, Carlsbad, Calif.) or XL1 Blue MR (Stratagene, La Jolla, Calif.) were screened by colony PCR, and clones containing inserts of the correct size were subjected to end sequencing to confirm the presence of the gene of interest and in-frame fusion to the appropriate reporter fragment. A subset of fusion constructs were selected for full-insert sequencing by primer walking. DNAs were isolated using Qiagen MaxiPrep kits (Qiagen, Chatsworth, Calif.). PCR was used to assess the integrity of each fusion construct, by combining the appropriate gene-specific primer with a reporter-specific primer to confirm that the correct gene-fusion was present and of the correct size with no internal deletions.

TABLE 1
Assays used in screening of cancer pathways in human cells

| Assay | Brief Assay Description |
|---|---|
| 14-3-3ζ: | CDC25 phosphatases regulate cell cycle |
| CDC25C | progression; 14-3-3 interaction indicates |
| | inactive phosphatase |
| Akt1:PDPK1 | Key node for insulin and apoptotic paths; |
| | increased signal and increased membrane |
| | localization indicates mitogenic and anti- |
| | apoptotic activity |
| Akt1: | Assessment of this signaling node in the |
| PDPK1 + | context of HGF stimulation |
| HGF | |
| Bad:BclxL | Key node for apoptotic signaling. Bad complexes |
| | with BclxL and Bcl-2 block the anti-apoptotic |
| | activity of the latter two proteins |
| BAD:BID | Indicates apoptotic activity |
| BAD:PAK4 | PAK phosphorylation of BAD is associated with |
| | decreased caspase activation and apoptosis |
| BIK:BCL-xL | Key node for apoptotic signaling. Bid complexes |
| | with BclxL and Bcl-2 block the anti-apoptotic |
| | activity of the latter two proteins |
| Cdc2: | Phosphatase/kinase complex; activity leads to |
| Cdc25A + | cell cycle progression |
| CPT | |
| CDC25C: | Phosphatase/kinase complex; activity leads to |
| Cdc2 + | cell cycle progression |
| CPT | |
| CDC25C:Cdc2 | Phosphatase/kinase complex; activity leads to |
| | cell cycle progression |
| CDC37:Hsp90 | HSP90 is key chaperone regulating protein |
| | stability/activity/half-life. CDC37 is co- |
| | chaperone; determines activity and client |
| | protein selectivity |
| CDC42:PAK4 | small GTPase/kinase signaling node. PAK4 is |
| | CDC42 effector; transmits the signal from the |
| | molecular switch to downstream substrates such |
| | as LIMK, BAD |
| Cdk2: | key cell cycle control node |
| CyclinE* | |
| Cdk4: | key cell cycle control node |
| CyclinD | |
| Chk1: | Chk kinases regulate CDC25 phosphatases; |
| CDC25C + | activation indicates cell cycle checkpoint |
| CPT | activation; CPT (camptothecin) topoisomerase |
| | inhibitor causes DNA damage and activates |
| | checkpoints |
| Chk1: | Chk kinases regulate CDC25 phosphatases; |
| CDC25A + | activation indicates cell cycle checkpoint |
| CPT | activation |
| Chk1:CDC25C | Chk kinases regulate CDC25 phosphatases; |
| | activation indicates cell cycle checkpoint |
| | activation |
| cofillin: | LIM kinases phospohorylate cofilin and regulate |
| LIMK2: | cytoskeletal dynamics |
| CyclinB: | mitotic complex; regulates APC |
| Cdc2* | |
| E6:E6AP* | papilloma virus E6 protein complexes with E6AP; |
| | and E3 ubiquitin ligase that targets p53 |
| E6:p53 | indicates p53 primed for proteasomal |
| | degradation |
| Eef2k:Hsp90 | translation factor-controlling kinase Eef2k is |
| | HSP client protein |
| EGFR:Grb2 | receptor tyrosine kinase:adaptor protein |
| | complex; indicates activated receptor |
| Erk2:Elk1 | ERK mitogen-activated protein kinase interacts |
| | with and phosphorylates the Elk-1 (Ets family) |
| | transcription factor |
| H-Ras:Raf* | small GTPase/kinase signaling node. Ras is |
| | commonly mutated human oncogene; activates |
| | ERK/MAP kinase path among others; downstream |
| | from receptor tyrosine kinases and some G- |
| | proteins |
| Hsp90:MEK1 | MEK is kinase upstream from ERK mitogen- |
| | activated protein kinases. It is an HSP90 |
| | client protein |
| MAPK9:ATF2 | MAPK9/JNK phosphorylates ATF-2 @ T72, |
| | activating its transcriptional activity |
| MKNK1:MAPK14 | MNK is a MAP kinase interacting and activated |
| | protein kinase |
| MYC:MAX | c-Myc is a transcription factor and human proto- |
| | oncogene. Activity correlates with cell cycle |
| | progression |
| p21:Cdc2 | p21 is cell cycle progression inhibitor, can |
| | exist in complex with Cdc2 and regulate |
| | activity |
| p27:MAPK1/ | p27 cell cycle inhibitor in complex with ERK |
| ERK2 | Map kinase |
| p53:Chk1 + CPT | Chk stimulates DNA-PK complex kinase activity, |
| | leading to p53 phosphorylation |
| p53:Chk1 | Chk stimulates DNA-PK complex kinase activity, |
| | leading to p53 phosphorylation |
| p53:p53 | increased interaction and dimerization of p53 |
| | indicates heightened activity of this node |
| p53:p53 + CPT | increased interaction and dimerization of p53 |
| | indicates heightened activity of this node |
| PAK4:Cofilin | complex of upstream activator PAK4 with |
| | downstream effector cofiin; regulates actin |
| | cytoskeleton |
| Pin1:CDC25C | prolyl isomerase Pin1 regulates conformation |
| | and activity of phosphatase CDC25C |
| Pin1:JUN* | prolyl isomerase Pin1 regulates conformation |
| | and activity of c-Jun, which in turn regulates |
| | cyclin D1 levels |
| Pin1:p53 | prolyl isomerase Pin1 regulates conformation |
| | and activity of p53 |
| PPARγ:RXRα | nuclear hormone receptor PPARgamma in typical |
| | active heterodimeric form |
| PPARγ:SRC-1 | nuclear hormone receptor PPARgamma in complex |
| | with specific nuclear transcription co- |
| | regulator |
| Rac1:Pak1 | small GTPase Rac in complex with its |
| | prototypical effector protein kinase PAK1 |
| RAD9:p38a | DNA damage response protein Rad9 in novel, |
| | functional complex |
| RAD9:p53 | DNA damage response protein Rad9 in novel, |
| | functional complex |
| Raf1:Map2k2 | Kinase Raf-1/subtrate MEK2 complex in |
| | Ras/MAPK path |
| RPS6K (70 | p70S6K/MKKK8 complex; growth factor |
| kDa):Map3k8 | stimulated and translational control |
| Smad3:HDAC | TGF beta responsive transcription factor Smad3 |
| | in nuclear with histone deacetylase |
| Wee1:Cdc2* | kinase Wee1 is negative regulator of Cdc2 (cell |
| | cycle progression kinase) |
| Akt1:p27* | Intersection of key anti-apoptotic (Akt) and |
| | cell cycle regulatory (p27) signaling nodes. |
| | Both targets involved in human tumors. |
| ESR1:SRC-1* | activity of estrogen receptor, and response to |
| | drugs, is dependent on regulated interactions |
| | with transcriptional co-factors including SRC-1 |
| p27:Ub* | p27 is key cell cycle regulator; loss is |
| | associated with human tumor progression. p27 |
| | levels are controlled by ubiquitination. |
| JUN:CBP | Immediate-early transcription factor and proto- |
| | oncogene c-Jun in transcriptionally active |
| | complex with CBP |
| p53:Mdm2 | Key human tumor gene in complex with it's |
| | negative regulator (Ub ligase Mdm2) |

Transfections and Cell Preparation

HEK293 cells were maintained in MEM alpha medium (Invitrogen) supplemented with 10% FBS (Gemini Bio-Products), 1% penicillin, and 1% streptomycin, and grown in a 37° C. incubator equilibrated to 5% CO₂. Approximately 24 hours prior to transfections cells were seeded into 96 well ploy-D-Lysine coated plates (Greiner) using a Multidrop 384 peristaltic pump system (Thermo Electron Corp., Waltham, Mass.) at a density of 7,500 cells per well. Up to 100 ng of the complementary YFP or IFP-fragment fusion vectors were co-transfected using Fugene 6 (Roche) according to the manufacturer's protocol. The list of the selected PCA pairs screened in this study, and corresponding gene and reporter fragment information, are listed in Table 2. Following 24 or 48 hours of expression, cells were screened against the selected drugs as described below.

For several PCAs, stable cell lines were generated. HEK293 cells were transfected with a first fusion vector and stable cell lines were selected using 100 μg/ml Hygromycin B (Invitrogen). Selected cell lines were subsequently transfected with the second, complementary fusion vector, and stable cell lines co-expressing the complementary fusions were isolated following double antibiotic selection with 50 μg/ml Hygromycin B and 500 μg/ml Zeocin. For all cell lines, the fluorescence signals were stable over at least 25 passages (data not shown). Approximately 24 hours prior to drug treatments, cells were seeded into 96 well ploy-D-Lysine coated plates (Greiner) using a Multidrop 384 peristaltic pump system (Thermo Electron Corp., Waltham, Mass.).

Drug Screening with PCA

Drugs were screened in duplicate wells at a concentration of 10 micromolar. All liquid handling steps were performed using the Biomek FX platform (Beckman Instruments, Fullerton, Calif.). Cells expressing the PCA pairs were incubated in cell culture medium containing drugs for 90 min. and 8 hours, or in some cases for 18 hours. For some assays cells were treated with known pathway agonists immediately prior to the termination of the assay. Following drug treatments cells were simultaneously stained with 33 micrograms/ml Hoechst 33342 (Molecular Probes) and 15 micrograms/ml TexasRed-conjugated Wheat Germ Agglutinin (WGA; Molecular Probes), and fixed with 2% formaldehyde (Ted Pella) for 10 minutes. Cells were subsequently rinsed with HBSS (Invitrogen) and maintained in the same buffer during image acquisition. YFP, Hoechst, and Texas Red fluorescence signals were acquired using the Discovery-1 automated fluorescence imager (Molecular Devices, Inc.) equipped with a robotic arm (CRS Catalyst Express; Thermo Electron Corp., Waltham, Mass.). The following filter sets were used to obtain images of 4 non-overlapping populations of cells per well: excitation filter 480/40 nm, emission filter 535/50 nm (YFP); excitation filter 360/40 nm, emission filter 465/30 nm (Hoechst); excitation filter 560/50 nm, emission filter 650/40 nm (Texas Red). All treatment conditions were run in duplicate yielding a total of 8 images for each wavelength and treatment condition.

Fluorescence Image Analysis

Raw images in 16-bit grayscale TIFF format were analyzed using ImageJ API/library (http://rsb.info.nih.gov/ij/, NIH, MD). First, images from all 3 fluorescence channels (Hoechst, YFP, and Texas Red) were normalized using the ImageJ built-in rolling-ball algorithm [S. R. Stemberg, Biomedical image processing. Computer, 16(1), January 1983]. Next a threshold was established to separate the foreground from background. An iterative algorithm based on Particle Analyzer from ImageJ was applied to the thresholded Hoechst channel image (HI) to obtain the total cell count. The nuclear region of a cell (nuclear mask) was also derived from the thresholded HI. A WGA mask was generated similarly from the thresholded Texas Red image. The positive particle mask was generated from the thresholded YFP image (YI). To calculate the global background (gBG), a histogram was obtained from the un-thresholded YI and the pixel intensity of the lowest intensity peak was identified as gBG. The Hoechst mask, WGA mask and YFP mask were overlapped to define the correlated sub-regions of the cell. The mean pixel intensity for all positive particles within each defined sub-region was calculated, resulting in 4 parameters: total positive pixel mean (MT, the mean intensity of the total particle fluorescence); Hoechst mean (M1, the mean intensity of the Hoechst defined region); Texas Red mean (M2, the mean intensity of the WGAdefined region); and Subtracted mean (M3, the mean intensity of the pixels excluded from the WGA- and Hoechst-defined regions). All means were corrected for the corresponding gBG.

For each set of experiments (assay+drug treatment+treatment time), all fluorescent particles from eight images were pooled. For each parameter, an outlier filter was applied to filter out those particles falling outside the range (mean±3SD) of the group. Next the sample mean or control mean for each parameter was obtained from each filtered group. For 7 out of 53 assays, we found it necessary to exclude low-intensity or autofluorescent particles from the analysis. We used a k-mean clustering algorithm [k=2, J. B. MacQueen, “Some Methods for Classification and Analysis of Multivariate Observations”, Proceedings of Berkeley Symposium on Mathematical Statistics and Probability, 1, Berkeley, Calif.: University of California Press, pp. 281-297, 1967] to separate the fluorescent particles into two populations, and used the cutoff derived from the control wells to exclude the lower-intensity population from the analysis.

Assay for Antiproliferative Activity

Human non-small cell lung carcinoma (A549, ATCC # CCL-185), colon adenocarcinoma (LoVo, ATCC # CCL-229), pancreatic carcinoma (MIA PaCa-2, ATCC # CRL-1420), prostate adenocarcinoma (PC-3, ATCC # CRL-1435), and glioblastoma (U-87 MG, ATCC # HTB-14) cells were acquired from American Type Culture Collection (ATCC, Manassas, Va.). Cells were maintained in various media as follows: A549, LoVo and PC-3 (Ham's F12K medium with 2 mM L-glutamine and 1.5 g/L sodium bicarbonate), MIA PaCa-2 (Dulbecco's modified Eagle's medium with 4 mM L-glutamine and 4.5 g/L glucose), U87-MG (MEM+Earle's BSS). Medium for each cell line was supplemented with 10% FBS and 100 mg/ml Penecillin/Streptomycin. All cells were grown in incubators set at 37° C., 5% CO₂. Thiazolyl Blue Tetrazolium Bromide (MTT) based proliferation assays were performed to assess the anti-proliferative activities of the compounds on these cells. Cells were seeded in 96 well plates at a density of 750 cells/well 24 hours prior to compound treatment. The cells were incubated with varying concentrations of compounds for 120 hours. Compound concentrations range from 0.03 to 100 microM (half log increments) except for alpha-Tomatine (0.001-100 microM, half log increments), Neriifolin (0.0002-100 microM) and Peruvoside (0.01-100 microM). Drug treatment was performed in 5 replicate wells. Background absorbance was established by wells containing medium but no cells. Vehicle (DMSO) only was used as control. MTT (Sigma-Aldrich, St. Louis, Mo.) was added to each well at a final concentration of 0.5 mg/ml. Following a 2 hour incubation at 37° C., medium in the wells was replaced with 0.15 ml DMSO. The plates were agitated for 15 min using a microtiter plate shaker. Absorbance at 560 nM was measured using SpectraMax Plus (Molecular Devices). Mean absorbance values were calculated from 5 replicate wells of each drug treatment following subtraction of background absorbance from blank samples and plotted as a percentage of control.

The invention also features a method for treating a patient having a neoplasm, said method comprising administering to said patient a therapeutic and effective amount of a drug selected from the group consisting of cinnarizine; desipramine; fenofibrate; flunarizine; reserpine; isoreserpine; nicardipine; promazine; promethazine; suloctidil; terfenadine; atorvastatin; mebeverine; sertraline; albendazole; bepridil; bergaptene; clomiphene; dichlorophene; droperidol; mebendazole; meclocycline; metergoline; ramiphenazone; sanguinarine; dipyrone; nicardipine; or 4-dimethylaminoantipyrine; or a metabolite or analog thereof; wherein said neoplasm is sensitive to said drug or a metabolite or analog thereof.

Unless otherwise indicated, the compounds useful for practicing the invention are meant to include pharmaceutically acceptable salts, prodrugs thereof, enantiomers, diastereomers, racemic mixtures thereof, crystalline forms, non-crystalline forms, amorphous forms thereof and solvates thereof.

The term pharmaceutically acceptable salts is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, phosphoric, partially neutralized phosphoric acids, sulfuric, partially neutralized sulfuric, hydroiodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present invention may contain both basic and acidic functionalities that allow the compounds to be converted into either base or acid addition salts.

The neutral forms of the compounds of the present invention may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

As noted above, some of the compounds useful in the practice of the present invention possess chiral or asymmetric carbon atoms (optical centers) or double bonds; the racemates, diastereomers, geometric isomers and individual optical isomers are all intended to be encompassed within the scope of the present invention.

Some of the compounds of useful in the practice of the present invention also exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

In addition to salt forms, the compounds useful in practicing the present invention may be in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex-vivo environment. For example, prodrugs can be slowly converted to the compounds of the present invention when placed in a transdermal patch reservoir with a suitable enzyme or chemical reagent.

In the present specification, the term therapeutically effective amount means the amount of the subject compound that will elicit the biological or medical response of a tissue, system, animal or human that is being sought by the researcher, veterinarian, medical doctor or other clinician.

The compounds useful for the practice of the invention can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They can be administered alone, but generally will be administered with a pharmaceutical carrier selected on the basis of the chosen route of administration and standard pharmaceutical practice.

The dosage regimen for the compounds useful in the present invention will, of course, vary depending upon known factors, such as the pharmacodynamic characteristics of the particular agent and its mode and route of administration; the metabolic stability, rate of excretion, drug combination, and length of action of that compound the species, age, sex, health, medical condition, and weight of the recipient; the nature and extent of the symptoms; the kind of concurrent treatment; the frequency of treatment; the specific route of administration, the renal and hepatic function of the patient, and the desired effect. A physician or veterinarian can determine and prescribe the effective amount of the drug required to prevent, counter, or arrest the progress of the specific disorder for which treatment is necessary.

Generally, the daily oral dosage of each active ingredient of the invention, when used for the indicated neoplasms, will range between about 0.0001 to 1000 mg/kg of body weight, preferably between about 0.001 to 100 mg/kg of body weight per day, and most preferably between about 0.1 to 20 mg/kg/day. For intravenous use, the most preferred doses will range from about 0.1 to about 10 mg/kg/minute during a constant rate infusion. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

The compounds useful in the instant invention can also be administered in intranasal form via topical use of suitable intranasal vehicles, or via transdermal routes, using transdermal skin patches. When administered in the form of a transdermal delivery system, the dosage administration-will, of course, be continuous rather than intermittent throughout the dosage regimen.

The compounds useful in the practice of the invention are typically administered in admixture with suitable pharmaceutical diluents, excipients, or carriers (collectively referred to herein as pharmaceutical carriers) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. For oral administration in liquid form, the oral drug components can be combined with any oral, non-toxic, pharmaceutically acceptable inert carrier such as ethanol, glycerol, water, and the like. Additionally, when desired or necessary, suitable binders, lubricants, disintegrating agents, and coloring agents can also be incorporated into the mixture. Suitable binders include starch, gelatin, natural sugars such as glucose or β-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention can also be provided to a patient in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or poly-ethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, and crosslinked or amphipathic block copolymers of hydrogels.

Dosage forms for the compounds useful for the invention and suitable for administration may contain from about 0.1 milligram to about 1000 milligrams of active ingredient per dosage unit. In these pharmaceutical compositions the active ingredient will ordinarily be present in an amount of about 0.5-95% by weight based on the total weight of the composition.

Gelatin capsules can also be used as dosage forms and may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

When using liquid dosage forms for oral administration they can contain coloring and flavoring to increase patient acceptance.

Generally, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in the field of pharmacology.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

The compounds useful in the practice of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. As used herein, topical application is also meant to include the use of mouth washes and gargles.

The pharmaceutical compositions and methods of the present invention may further comprise other therapeutically active compounds which are usually applied in the treatment of the above mentioned neoplastic conditions.

TABLE 2
Drugs found to have anti-cancer-pathway activity and anti-proliferative activity

| Compound | | | FDA Patent/ | | | | |
|---|---|---|---|---|---|---|---|
| Name | Synonyms | Merck Ref# | Exclusivity | Pathway Activity | Tumor Cell | IC-50 | Original Indication |
| Albendazole | Albenza | Merck | Expired | invasion/metastasis | NSCLC | 0.28 | antihelmintic |
| | | 13,209 | | | Colon | | |
| | | | | | Pancreatic | 0.14 | |
| | | | | | Prostate | | |
| | | | | | Glioblastoma | | |
| Mebendazole | Vermox | Merck | Expired | invasion/metastasis | NSCLC | 0.18 | antihelmintic |
| | | 13,5791 | | | Colon | | |
| | | | | | Pancreatic | 0.13 | |
| | | | | | Prostate | | |
| | | | | | Glioblastoma | | |
| Bepridil | Bepadin | Merck | Expired | invasion/metastasis | NSCLC | 4.74 | Angina |
| | Vascor | 13,1153 | | | Colon | | |
| | | | | | Pancreatic | 1.58 | |
| | | | | | Prostate | | |
| | | | | | Glioblastoma | | |
| Cinnarizine | Stugeron | Merck | None | apoptosis | NSCLC | 5.71 | antiemetic |
| | | 13,2328 | | | Colon | 7.14 | |
| | | | | | Pancreatic | >40 | |
| | | | | | Prostate | 10.5 | |
| | | | | | Glioblastoma | >40 | |
| Fenofibrate | Tricor | Merck | Expired | apoptosis | NSCLC | 41.6 | hypercholesterolemia |
| | Lipidil | 13,4005 | | | Colon | 19.05 | hypertriglyceridemia |
| | | | | | Pancreatic | 5.24 | |
| | | | | | Prostate | 34.3 | |
| | | | | | Glioblastoma | 40 | |
| Sanguinarine | | Merck | None | proliferation | NSCLC | 2.23 | Dental Hygeine |
| | | 13,8433 | | | Colon | | |
| | | | | | Pancreatic | 1.93 | |
| | | | | | Prostate | | |
| | | | | | Glioblastoma | | |
| Peruvoside | | | None | cell cycle | NSCLC | <0.03 | Congestive heart failure |
| | | | | | Colon | | |
| | | | | | Pancreatic | <0.03 | |
| | | | | | Prostate | | |
| | | | | | Glioblastoma | | |
| Neriifolin | | Merck | None | invasion/metastasis | NSCLC | 0.006 | Congestive heart failure |
| | | 13,6499 | | | Colon | | |
| | | | | | Pancreatic | 0.003 | |
| | | | | | Prostate | | |
| | | | | | Glioblastoma | | |
| Isoreserpine | | | None | apoptosis | NSCLC | 3.95 | hypertension |
| | | | | | Colon | 2.73 | |
| | | | | | Pancreatic | 3.92 | |
| | | | | | Prostate | 11.7 | |
| | | | | | Glioblastoma | 9.02 | |
| Reserpine | Hiserpia | Merck | Expired | (analog of PCA hit) | NSCLC | | hypertension |
| | RAU-SED | 13,8231 | | | Colon | | |
| | Sandril | | | | Pancreatic | | |
| | Serpalan | | | | Prostate | | |
| | Serpanray | | | | Glioblastoma | | |
| | Serpasil | | | | | | |
| | Serpate | | | | | | |
| | Serpivite | | | | | | |
| Niclosamide | Niclocide | Merck | Expired | apoptosis | NSCLC | 0.66 | anthelmintic |
| | | 13,6543 | | | Colon | 0.45 | |
| | | | | | Pancreatic | 0.59 | |
| | | | | | Prostate | 1.33 | |
| | | | | | Glioblastoma | 0.81 | |
| Promazine | Sparine | Merck | Expired | proliferation | NSCLC | 11.92 | Sedative |
| | Prozine | 13,7874 | | | Colon | | Antipsychotic |
| | | | | | Pancreatic | 1.97 | |
| | | | | | Prostate | | |
| | | | | | Glioblastoma | | |
| Terfenadine | Seldane | Merck | Expired | invasion/metastasis | NSCLC | 0.55 | Allergies |
| | | 13,9239 | | | Colon | 0.99 | |
| | | | | | Pancreatic | 0.35 | |
| | | | | | Prostate | 1.32 | |
| | | | | | Glioblastoma | 0.38 | |
| Clomiphene | Clomid | Merck | Expired | proliferation | NSCLC | 8.35 | olulatory disfunction |
| | Milophene | 13,2410 | | | Colon | | |
| | Serophene | | | | Pancreatic | 3.0 | |
| | | | | | Prostate | | |
| | | | | | Glioblastoma | | |
| Dichlorophene | | Merck | None | proliferation | NSCLC | 9.83 | antihelmintic |
| | | 13,3096 | | | Colon | | antiprotozoan |
| | | | | | Pancreatic | 25.8 | |
| | | | | | Prostate | | |
| | | | | | Glioblastoma | | |
| Droperidol | Inapsine | Merck | Expired | invasion/metastasis | NSCLC | 29.44 | Adjunct with Anesthesia; |
| | | 13,3484 | | | Colon | | antiemetic |
| | | | | | Pancreatic | 13.92 | antipsychotic |
| | | | | | Prostate | | |
| | | | | | Glioblastoma | | |
| Mebendazole | Vermox | Merck | Expired | invasion/metastasis | NSCLC | 0.28 | anthelmintic |
| | | 13,5791 | | | Colon | | |
| | | | | | Pancreatic | 0.29 | |
| | | | | | Prostate | | |
| | | | | | Glioblastoma | | |
| Meclocycline | Meclan | Merck | Expired | invasion/metastasis | NSCLC | 18.76 | antibiotic |
| | | 13,5801 | | | Colon | | |
| | | | | | Pancreatic | 68.21 | |
| | | | | | Prostate | | |
| | | | | | Glioblastoma | | |
| Melergoline | none | Merck | None | invasion/metastasis | NSCLC | 5.7 | Hyperprolactinaemia |
| | | 13,5962 | | | Colon | | |
| | | | | | Pancreatic | 20.74 | |
| | | | | | Prostate | | |
| | | | | | Glioblastoma | | |
| Tomatine | Lycopersicin | Merck | None | invasion/metastasis | NSCLC | 0.06 | Adjuvant |
| | | 13,9623 | | | Colon | | |
| | | | | | Pancreatic | 0.1 | |
| | | | | | Prostate | | |
| | | | | | Glioblastoma | | |
| Methoxsalen | 8-MOP | Merck | Expired | (analog of PCA | NSCLC | | photochemotherapy |
| | Oxsoralen | 13,6018 | | hit Jun. 1, 2004 | Colon | | (psoriasis, vitiligo, cutaneous |
| | Uvadex | | | Bergapteneanalog) | Pancreatic | | T-cell lymphoma) |
| | | | | | Prostate | | |
| | | | | | Glioblastoma | | |
| Ramifenazone | Isopryn | Merck | None | invasion/metastasis | NSCLC | | Migraine |
| | | 13,8193 | | | Colon | | |
| | | | | | Pancreatic | | |
| | | | | | Prostate | | |
| | | | | | Glioblastoma | | |
| Atorvastatin | Lipitor | Merck | Protected | invasion/metastasis | NSCLC | >40 | Hypercholesteremia |
| | | 13,868 | | | Colon | 16.95 | |
| | | | | | Pancreatic | 6.09 | |
| | | | | | Prostate | 10.6 | |
| | | | | | Glioblastoma | 4.6 | |
| beta-Lapachone | | | None | apoptosis | NSCLC | 0.23 | sepsis: has known anti- |
| | | | | | Colon | 1.4 | cancer activity |
| | | | | | Pancreatic | 0.77 | |
| | | | | | Prostate | 0.63 | |
| | | | | | Glioblastoma | 0.86 | |
| Mebeverine | Colofac | Merck | None | invasion/metastasis | NSCLC | | GI Spams, IBS |
| | | 13,5792 | | | Colon | | |
| | | | | | Pancreatic | | |
| | | | | | Prostate | | |
| | | | | | Glioblastoma | | |
| Sertraline | Zoloft | Merck | Protected | apoptosis | NSCLC | 1.22 | depression |
| | | 13,8541 | | | Colon | 2.79 | |
| | | | | | Pancreatic | 0.87 | |
| | | | | | Prostate | 4.46 | |
| | | | | | Glioblastoma | 7.4 | |
| Desipramine | Norpramine | Merck | Expired | proliferation | NSCLC | 11.88 | depression |
| | Pertofrane | 13,2937 | | | Colon | | |
| | | | | | Pancreatic | 1.93 | |
| | | | | | Prostate | | |
| | | | | | Glioblastoma | | |
| Flunarizine | Sibelium | Merck | None | invasion/metastasis | NSCLC | 18.23 | prophylaxis of migraine |
| | | 13,4170 | | | Colon | | |
| | | | | | Pancreatic | 22.53 | |
| | | | | | Prostate | | |
| | | | | | Glioblastoma | | |
| Nicardipine | Cardene | Merck | Expired | proliferation | NSCLC | 21.86 | hypertension |
| (Cardine) | | 13,6520 | | | Colon | | |
| | | | | | Pancreatic | 20.41 | |
| | | | | | Prostate | | |
| | | | | | Glioblastoma | | |
| Promethazine | Phenergan | Merck | Expired | proliferation | NSCLC | 53.82 | Sedative |
| | | 13,7878 | | | Colon | | Antipsychotic |
| | | | | | Pancreatic | 22.18 | |
| | | | | | Prostate | | |
| | | | | | Glioblastoma | | |
| Suloctidil | Sulocton | Merck | None | invasion/metastasis | NSCLC | 0.78 | dementia |
| | | 13,9077 | | | Colon | | antithrombotic |
| | | | | | Pancreatic | 0.18 | |
| | | | | | Prostate | | |
| | | | | | Glioblastoma | | |
| Bergaptene | | Merck | None | proliferation; invasion/ | NSCLC | 56.4 | dermatoses |
| | | 13,1160 | | metastasis | Colon | | |
| | | | | | Pancreatic | 58.3 | |
| | | | | | Prostate | | |
| | | | | | Glioblastoma | | |

The vehicle (DMSO) used to deliver the compounds to the cells had little or no effect on proliferation in the same assays.

The antiproliferative effects demonstrated with the tumor cells tested herein can be similarly demonstrated using other cancer cell lines, such as MCF7 mammary adenocarcinoma, PA-1 ovarian teratocarcinoma, HT20 colorectal adenocarcinoma, H1299 large cell carcinoma, U-20S osteogenic sarcoma, Hep-3B hepatocellular carcinoma, BT-549 mammary carcinoma, T-24 bladder cancer, C-33A cervical carcinoma, HT-3 metastatic cervical carcinoma, SiHa squamous cervical carcinoma, CaSki epidermoid cervical carcinoma, NCI-H292 mucoepidermoid lung carcinoma, NCI-2030 non small cell lung carcinoma, HeLa epithelial cervical adenocarcinoma, KB epithelial mouth carcinoma, HT1080 epithelial fibrosarcoma, Saos-2 epithelial osteogenic sarcoma, SW480 colorectal carcinoma, CCL-228 and MS-751 epidermoid cervical carcinoma cell lines. The specificity can be tested by using cells such as NHLF lung fibroblasts, NHDF dermal fibroblasts, HMEC mammary epithelial cells, PrEC prostate epithelial cells, HRE renal epithelial cells, NHBE bronchial epithelial cells, PrEC prostate epithelial cells, HRE renal epithelial cells, NHBE bronchial epithelial cells, CoSmC colon smooth muscle cells, CoEC colon endothelial cells, NHEK epidermal keratinocytes, and bone marrow cells as control cells.

We are advancing many of these candidates into preclinical and clinical trials in cancer patients.

The entire contents including the references cited therein of the following patents and publications are incorporated by reference in their entirety for all purposes to the same extent as if each individual patent, patent application or publication were so individually denoted.
U.S. Pat. No. 6,372,431 Cunningham, et al.
U.S. Pat. No. 6,801,859 Friend, et al.
U.S. Pat. No. 6,673,554 Kauvar, et al.
U.S. Pat. No. 6,270,964 Michnick, et al.
U.S. Pat. No. 6,294,330 Michnick, et al.
U.S. Pat. No. 6,428,951 Michnick, et al.
U.S. Patent Application 20030108869 Michnick, et al.
U.S. Patent Application 20020064769 Michnick, et al.

Although the present invention has been described with reference to specific details of certain embodiments thereof, it is not intended that such detail should be regarded as limitations upon the scope of the invention, except as and to the extent that they are included in the accompanying claims.

What is claimed is:

1. A method for treating a mammal patient having a neoplasm, said method comprising administering to said patient an effective amount of drug selected from the group consisting of cinnarizine; desipramine; fenofibrate; flunarizine; reserpine; isoreserpine; nicardipine; promazine; promethazine; suloctidil; terfenadine; atorvastatin; mebeverine; sertraline; albendazole; bepridil; bergaptene; clomiphene; dichlorophene; droperidol; mebendazole; meclocycline; metergoline; ramiphenazone; sanguinarine; dipyrone; nicardipine; or 4-dimethylaminoantipyrine; or a metabolite or analog thereof; wherein said neoplasm is sensitive to said drug or a metabolite or analog thereof.

2. A method for treating a mammal patient having a neoplasm, said method comprising administering to a patient a compound selected from the group consisting of neriifolin, peruvoside, tomatine, or niclosamide, or a metabolite or an analog thereof.

3. The method of claim 1, wherein said neoplasm is a cancer.

4. The method of claim 2, wherein said neoplasm is a cancer.

5. The method of claim 2, wherein said method is performed in conjunction with administering to said patient an additional treatment for cancer, wherein said additional treatment is administered within 6 months of said treatment.

6. The method of claim 2, said additional treatment comprising surgery, radiation therapy, chemotherapy, immunotherapy, anti-angiogenesis therapy, gene therapy, interfering RNA therapy, vaccine therapy.

7. The method of claim 4, wherein said additional treatment is selected from: bleomycin, carmustine, cisplatin, daunonibicin, etoposide, melphalan, mercaptopurine, methotrexate, mitomycin, vinblastine, paclitaxel, docetaxel, vincristine, vinorelbine, cyclophosphamide, chlorambucil, gemcitabine, capecitabine, 5-fluorouracil, fludarabine, raltitrexed, irinotecan, topotecan, doxorubicin, epirubicin, letrozole, anastrazole, formestane, exemestane, tamoxifen, toremefine, goserelin, leuporelin, bicalutamide, flutamide, tamoxifen, (GNRH ANTAGONIST), nilutamide, hypericin, trastuzumab, rituximab, (iressa, other) or any combination thereof.

8. The method of claim 2, wherein said cancer is selected from the group consisting of acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkins disease, chondrosarcoma, osteogenic sarcoma, chordoma, angiosarcoma, endotheliosarcoma, lymphagiosarcoma, lymphagioendotheliosarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarconoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, astrocytoma, craniopharyngioma, ependymoma, pinealoma, hemangioblastoma, acoustic neuroma, oligodendriglioma, schwannoma, meningioma, melanoma, neuroblastoma, and retinoblastoma.

9. The method of claim 2, wherein said cancer is lung cancer.

10. The method of claim 8, wherein said lung cancer is non-small-cell lung cancer, adenocarcinoma, small-cell lung cancer.

11. The method of claim 2, wherein said cancer is colon cancer or rectal cancer.

12. The method of claim 2, wherein said cancer is pancreatic cancer.

13. The method of claim 2, wherein said cancer is prostate cancer.

14. The method of claim 2, wherein said cancer is glioblastoma.

15. The method of claim 2, wherein said cancer is ovarian cancer.

16. The method of claim 2, wherein said cancer is breast cancer.

17. The method of claim 2, wherein said composition is administered to said patient by intravenous, intramuscular, inhalation, rectal, topical, or oral administration.

18. A composition, wherein a drug selected from the group consisting of cinnarizine, desipramine, fenofibrate, flunarizine, isoreserpine, reserpine, nicardipine, promazine, promethazine, suloctidil, terfenadine, atorvastatin, mebeverine, sertraline, albendazole, bepridil, bergaptene, clomiphene, dichlorophene, droperidol, mebendazole, meclocycline, metergoline, ramiphenazone, sanguinarine, dipyrone, nicardipine, or 4-dimethylaminoantipyrine, is present in amounts that, when administered to a patient having a neoplasm, reduce cell proliferation in said neoplasm.

19. A composition, wherein a compound selected from the group consisting of: neriifolin, peruvoside, tomatine, or niclosamide are present in amounts that, when administered to a patient having a neoplasm, reduce cell proliferation in said neoplasm.
